(19) United States
(12) Patent Application Publication
(10) Pub. No.: US 2002/0176796 A1
(43) Pub. Date: Nov. 28, 2002

(54) Inactivation of microbes in biological fluids

(75) Inventors: Jeffrey Morse Holloway, San Diego, CA (US); Kenton Salisbury, San Diego, CA (US); William H. Cover, Escondido, CA (US); Mark Landers, San Diego, CA (US)

Correspondence Address: FITCH EVEN TABIN AND FLANNERY, 120 SOUTH LA SALLE STREET, SUITE 1600, CHICAGO, IL, 60603-3406, US

(73) Assignee: PurePulse Technologies, Inc.

(21) Appl. No.: 10/067,731

(22) Filed: Feb. 4, 2002

Related U.S. Application Data
(63) Continuation-in-part of application No. 09/596,987, filed on Jun. 20, 2000.

(51) Int. Cl. A61L 2/00; A62B 7/08; B01J 19/08

(52) U.S. Cl. 422/24; 422/121; 422/186.3

(57) ABSTRACT

The present invention relates to methods and treatment systems for inactivation of microbes and/or nucleic acids in biological fluids, especially platelet compositions without completely damaging antigens, enzymes and membrane functions. In accordance with the method of the invention, a biological fluid is illuminated with a light source having at least one wavelength within a range of 170 to 2600 nm to inactivate microbes in the composition and inactivate nucleic acids inside cells without destroying proteins (enzymes) and membrane functions.

This application is a continuation-in-part application of U.S. application Ser. No. 09/596,987, filed Jun. 20, 2000, which is incorporated herein in its entirety by reference.

### FIELD OF INVENTION

The present invention relates to methods and treatment systems for inactivation of microbes and/or nucleic acids in biological fluids, especially platelet compositions without completely damaging antigens, enzymes and membrane functions. More particularly, the methods and systems of the present invention utilize illumination of platelet compositions with a light source having at least one wavelength within a range of 170 to 2600 nm to inactivate microbes in the platelet composition and inactivate nucleic acids inside cells without destroying proteins (enzymes) and membrane functions.

### BACKGROUND

Biological fluids used in connection with human therapies are required to meet certain criteria prescribed by regulatory agencies in terms of purity and contaminant levels. Substantial technical efforts have been directed to inactivating contaminating nucleic acids and microbes in biological fluids.

A biological fluid of particular interest is platelets. Platelets are disk-shaped blood cells that are also called thrombocytes. They play a major role in the blood-clotting process. Platelets can be harvested from single donors by plateletapheresis or separated from whole blood, with pooling of cells from multiple donors to achieve a therapeutic dose. Single-donor platelets can be obtained from random donors or from donors selected on the basis of HLA compatibility.

In the past, patients with chronic thrombocytopenia died of hemorrhage with distressingly predictable frequency. The increased use of platelet transfusions during the past 15 years has prevented most such deaths. Furthermore, this therapy has made it possible to treat patients with drugs who have otherwise fatal disorders that temporarily suppress platelet production. With this great benefit, however, have come complex problems. Transfused platelets can transmit fatal diseases and can elicit an immune response in recipients, so that further transfusions are no longer effective.

A high percentage of platelet rich plasma units are contaminated with bacteria and/or virus. The actual collection process itself introduces bacterial and viral pathogens. Currently there are no FDA approved pathogen inactivation methods for the decontamination of platelet rich plasma or red blood cells.

Platelet rich plasma is stored at room temperature for up to 5 days. The room temperature storage and high nutrient content of platelet units represent a good culture medium for bacteria present upon apherisis. The high content of bacteria in human skin represent a significant infection risk upon apherisis, via the skin plug. A single bacterial cell present upon apherisis collection can proliferate into 10⁷ CFU's/mL before the 5 day expiration date is reached.

Currently bacterial detection methods are not sensitive enough to detect low levels of bacterial contamination upon collection. Most detection methods are only specific for certain species of bacteria. Therefore, if a platelet unit is not screened for all possible blood contaminates immediately before transfusion the possibility of infecting the recipient with a pathogenic organism is significant. Further, testing would be impractical due to the 1-3 day testing turn around time for detection of microbe contamination.

Pathogen inactivation in platelet rich plasma is difficult due to the high concentration of leukocytes, as well as platelets, in the solution. Inactivating pathogens without damaging the platelets ability to function in the clotting cascade is difficult due to the similarity of the pathogens to the platelets as well as the chronic association of bacteria and virus to the platelet and leukocyte cell walls. Platelets contain no nucleic acids, unlike most pathogens, subsequently most inactivation technologies used in platelets target nucleic acids. Currently several chemical and photochemical treatments have been used to treat platelet rich plasma and red blood cell solutions. Psoralen (Cerus) and Riboflavin (Gambro) technologies require photoactivation to stimulate the chemicals irreversible binding to nucleic acids. Inactine (Vitex) compounds also bind nucleic acids but do not require photoactivation. To be effective the chemical must penetrate the cell wall and bind to the pathogen's nucleic acids. Many pathogen cell walls do not allow the chemical to penetrate therefore limiting the selectivity of the chemical agent. In addition, many chemical inactivation compounds are genotoxic to humans upon transfusion.

### SUMMARY

The present invention provides a fast, reliable and efficient method for the inactivation of microbes and endogenous nucleic acids and/or nucleic acid strands in biological fluids. The method of the invention is effective for inactivating nucleic acids located inside the cytoplasm of a cell wall without destroying the functionality of other macromolecules, membranes, cell walls, antigens, enzymes and cell functions. Further, the method of the invention is effective for inactivation of endogenous nucleic acid strands without causing a decrease in cellular metabolic activity of the cell containing the nucleic acid strand.

In one important aspect, the present invention provides a method for inactivating microbes in platelet compositions, thus improving the safety and shelf life of platelet compositions. The use of bright spectrum pulsed light (BSPL) in accordance with the present invention is effective for inactivating microbes in the platelet compositions while not inactivating the biological functions of the platelets. The method of inactivating microbes of the present invention does not add possibly genotoxic chemical agents and does not effect the platelets ability to aggregate and participate in the formation of a blood clot. BSPL treatment in accordance with the present invention does not result in any significant changes to platelet morphology and physiology.

In accordance with the method of the invention, platelet composition is illuminated with pulses of light having at least one wavelength within a range of 170 to 2600 nm either in a batch process or continuous process. The process of the invention is effective for extending the shelf life of platelet composition by at least about 4 days as compared to platelet composition that have not been exposed to a light treatment having at least one wavelength within a range of 170 to 2600 nm. The exposure of platelet compositions according to the present invention is effective for inactivating microbes in the platelet composition while not causing extensive protein damage or inactivation of platelet function. In this aspect of the invention, platelet aggregation after BSPL treatment at the fluence levels described is not decreased more than about 40%.

In an important aspect of the invention, the fluence or intensity of the pulsed light is from about 0.001 J/Cm² to about 50 J/cm². The fluence of the pulsed light is carefully selected to avoid extensive protein damage or inactivation of platelets while at the same time inactivating microbes to a specified log reduction. For example, platelet compositions may be illuminated with about 2 to about 100 pulses of light having a duration of less than about 100 ms effective for providing a fluence level preferably between about 0.01 and to about 15 J/cm² (about 0.05 to about 1.5 J/flash).

In one aspect of the present invention, microbes in a platelet composition are inactivated by flowing the platelet composition through a treatment chamber being light transmissive to at least 1% of a light treatment having at least one wavelength within a range of 170 to 2600 nm. The platelet composition is illuminated with the light as the platelet composition is flowed through the treatment chamber. Illumination is effective for reducing any microbes in the platelet composition by at least about 2 logs and for preventing any increase in microbial levels in the biological fluid for at least about 4 to about 6 days.

In another aspect of the invention, microbes in a platelet composition may be inactivated by treating the platelet composition in a batch mode. In this aspect, the platelet composition being treated may be placed into its final container, such as for example an IV bag, and then illuminated with light having at least one wavelength within a range of 170 to 2600 nm.

In another aspect of the invention, platelet compositions may be illuminated periodically over time in an amount effective to maintain any microbes in the platelet composition in an inactive state. In accordance with this aspect of the invention, the platelet composition may be illuminated every 6 hours with light having at least one wavelength within a range of 170 to 2600 nm at a fluence level effective for maintaining microbes in an inactive state and inhibiting any increase in microbial counts in the platelet composition.

In another important aspect, the invention provides a method for inactivating an endogenous nucleic acid strand which may be in a biological fluid. In accordance with the method of the invention, the cell containing the endogenous nucleic acid strand or the biological fluid that include the endogenous nucleic acid strand is exposed to a broad-spectrum pulsed light treatment as described above either in a batch process or continuous process. Exposure of the cell or biological fluid that includes the endogenous nucleic acid strand results in an inactivation of nucleic acid strands as compared to cell and biologial fluids that have not been exposed to BSPL. In an important aspect, nucleic acid strands are inactivated to a level where they are no longer a concern for regulatory purposes or interfere with various types of assays such as PCR. In another aspect of the invention, the inactivation of nucleic acid strands does not result in elimination of cellular metabolic activity in the cell containing the nucleic acid strand or an elimination of the overall biological activity of the biological fluid. The present invention allows the inactivation of nucleic acids and recovery of cellular function.

### BRIEF DESCRIPTION OF THE DRAWINGS

The above and other aspects, features and advantages of the present invention will be more apparent from the following more particular description thereof, presented in conjunction with the following drawings wherein:

FIG. 1 is a graph of BSPL E. coli kill curves in various dilutions of concentrated platelets exposed to various levels of BSPL at a 5 mm sample depth using multiple fluence/flash levels;

FIG. 2 is a graph showing S. epidermidis survivors after periodic treatment of S. epidermidis spiked concentrated platelet solution with 10 flashes of 0.5 J/F BSPL every hour for 4 hours in a 1.5 mm flat plate with mixing;

FIG. 3A is a graph BSPL S. epidermis kill curves of concentrated platelet solutions spiked with 102 bacteria and treated with 10 flashes of 0.25 J/F every 2 hours for a total of six hours;

FIG. 3B is a graph BSPL S. epidermis kill curves of concentrated platelet solutions spiked with 10⁴ bacteria and treated with 10 flashes of 0.25 J/F every 2 hours for a total of six hours;

FIG. 3C is a graph BSPL S. epidermis kill curves of concentrated platelet solutions spiked with 10⁶ bacteria and treated with 10 flashes of 0.25 J/F every 2 hours for a total of six hours;

FIG. 4 is a graph showing % aggregation of a 1:10 dilution of platelets with various levels of BSPL treatment;

FIG. 5 is a graph showing the number of surviving cells after periodic BSPL treatment of platelet rich plasma spiked with S. epidermis treated with 5 J of energy (10 flashes at 0.5 J/F) every 2 hours for 6 hours;

FIG. 6 is a graph showing results of in-flow (1 mm sample depth at 50 ml/min) BSPL treatment of platelet rich plasma spiked with S. epidermis using 0.25 J/F BSPL with 0 to 2.0 J/cm²;

FIG. 7 is a graph illustrating relative platelet aggregation vs. total energy BSPL in platelet rich plasma diluted 1:10 with PAS III;

FIG. 8 is a graph illustrating percentage platelet aggregation after BSPL treatment of platelet rich plasma after a 1:10 dilution with PAS III and reconstitution with fetal bovine serum;

FIG. 9 is a graph showing plasma glucose levels following exposure to BSPL; and

FIG. 10 is a graph showing plasma lactic acid levels following exposure to BSPL.

### DETAILED DESCRIPTION

The present invention advantageously addresses the needs above as well as other needs by providing a treatment method for the inactivation of microbes and nucleic acid strands in biological fluids, especially platelet compositions.

Inactivation of Microbes and Nucleic Acids

The methods of the present invention are effective for inactivation of microbes. As used herein “microbes” refers to bacteria, viruses and fungi. Examples of bacteria know to sometimes be a contaminant in biological fluids and in platelet compositions which may be inactivated by the methods of the present invention include for example E. coli, S. epidermidis, Staphlococcus sp., Streptococcus sp., S. pneumoniae, Bacillus sp., Pseudomonas sp., Cornebacterium sp., Neisseria sp., Neisseria meningitidis, Neisseria gonorrhoeae, and Clostridium sp. Examples of viruses known to sometimes be a contaminant in biological fluids and in platelet compositions which may be inactivated by the methods of the present invention include for example adenoviruses, herpesviruses, poxviruses, pirconaviruses, orthomyxoviruses, paramyxoviruses, cornoaviruses, rhabdoviruses, HIV, and hepatitis viruses. Examples of fungi known to sometimes be a contaminant in biological fluids and in platelet compositions which may be inactivated by the methods of the present invention include for example fungi or the class phycomycetes, ascomycetes, basidiomycetes and deuteromycetes.

As used herein, “inactivation of microbes” refers to a reduction of microbial counts in a biological fluid of at least about 2 logs or a zero net increase of microbial counts in a biological fluid for at least about 4 to about 6 days after treatment. A biological fluid may be illuminated periodically to reduce and/or maintain microbial counts at a desired level.

As used herein, “inactivation of nucleic acids” refers to a method of forming inactive nucleic acids through treatment with BSPL. In order for the nucleic acid to be considered inactive, or an “inactive nucleic acids” the nucleic acids must not be suitable for replication, amplification, or translation. Generally, this will mean that the inactive nucleic acid is not a suitable template for a polymerase as the nucleic acid is too short to serve as a template. Hence, inactive nucleic acids will not be capable of interfering with a PCR assay as they will not replicate or amplify, or not replicate or amplify to a level that would interfere with the assay. Further, an inactive nucleic acid may be degraded, cleaved or neutralized to an extent that it no longer can function biologically as it did prior to treatment with BSPL.

In an important aspect, the method of the invention is effective for inactivating endogenous nucleic acids. As used herein, “endogenous nucleic acids and endogenous nucleic acid strands” are nucleic acids and nucleic acid strands that occur within a cellular membrane. The method of the present invention is effective for inactivating endogenous nucleic acids without inactivating the biological function of the cell that contain the endogenous nucleic acids. For inactivation of endogenous nucleic acids without a decrease in biological function of the cell containing the endogenous nucleic acids, the cell may be illuminated with about 1 to about 50 pulses of light having a duration of less than about 100 ms which are effective for providing a fluence level between about 0.005 to about 10 J/cm².

Biological Fluids

As used herein, the term “biological fluids” refer to pharmaceutical compositions and compositions such as platelet compositions, vaccines, plasma, monoclonal antibodies, protein from genetically engineered mammalian cell lines, gene therapy products, human and/or animal blood derived products, plant derived compositions, hormones, gelatin, biological pharmaceutics such as heparin and/or collagen, bovine serum, sheep blood, peptones/amino acids and/or bovine insulin/transferrin, fermentation broths and mixtures thereof.

As used herein, the term “platelet compositions” include platelet rich plasma, leukocyte reduced platelets, non-leukocyte reduced platelets, synthetic platelet substitutes, artificial platelets, recombinant platelet products, and mixtures thereof. The biological fluids of the invention may be placed into their final container prior to illumination. For example, platelet compositions, may be placed into an IV bag prior to illumination.

In accordance with the present invention, platelet compositions are treated primarily to inactivate microbes without causing excessive protein damage or inactivation of platelet function. Thus, in this aspect of the invention, the pulsed light treatment is configured to provide greater than about 2 logs reduction, more preferably greater than about 4 logs reduction and most preferably greater than about 6 logs reduction is achieved with minimum protein damage or inactivation of platelet function. Although some of these deactivation levels fall short of what is accepted as sterilization, the pulsed light provides a significant advantage over a continuous wave UV treatment system in that pathogens and other contaminants are effectively deactivated at desired log reduction rates with minimum protein damage or inactivation of platelet function in a short period of time. As describe herein, platelet functionality may be measured by determining platelet aggregation, plasma glucose levels or plasma lactic acid levels after BSPL treatment. In this aspect, BSPL treatment is effective for decreasing platelet aggregation by not more than about 40%, decreasing plasma glucose levels by not more than about 5%, or for decreasing plasma lactic acid levels by not more than about 5%.

Broad-Spectrum Pulsed Light

Broad-spectrum pulsed light (BSPL) described through this specification may also be referred to generically as “pulsed polychromatic light” or even more generically as pulsed light. Pulsed polychromatic light represents pulsed light radiation over multiple wavelengths. For example, the pulsed polychromatic light may comprise light having wavelengths between 170 nm and 2600 nm inclusive, such as between 180 nm and 1500 nm, between 180 nm and 1100 nm, between 180 nm and 300 nm, between 200 and 300 nm, between 240 and 280 nm, or between any specific wavelength range within the range of 170-2600 nm, inclusive.

As is generally known, Xenon gas flashlamps produce pulsed polychromatic light having wavelengths at least from the far ultraviolet (200-300 nm), through the near ultraviolet (300-380 nm) and visible (380 nm-780 nm), to the infrared (780-1100 nm). In one example, the pulsed polychromatic light produced by these Xenon gas flashlamps is such that approximately 25% of the energy distribution is ultraviolet (UV), approximately 45% of the energy distribution is visible, and approximately 30% of the energy distribution is infrared (IR) and beyond. It is noted that the fluence or energy density at wavelengths below 200 nm is negligible, e.g., less than 1% of the total energy density. Furthermore, these percentages of energy distribution may further be adjusted. In other words, the spectral range may be shifted (e.g., by altering the voltage across the flashlamp) so that more or less energy distribution is within a certain spectral range, such as UV, visible and IR. In some embodiments it may be preferable to have a higher energy distribution in the UV range. Furthermore, the use of BSPL using Xenon flashlamps completely eliminates the problem of Mercury contamination due to broken Mercury lamps that may be encountered in such a continuous wave UV fluid treatment device, since Xenon is an inert gas which is harmless if exposed due to leakage or breaking of the Xenon flashlamp. Variants of Xenon flashlamps, such as those described in U.S. Pat. No. 6,087,783 of Eastland, et al., entitled METHOD AND APPARATUS UTLILIZING MICROWAVES TO ENHANCE ELECTRODE ARC LAMP EMISSION SPECTRA, issued Jul. 11, 2000, which is incorporated herein by reference, may also be used as an appropriate light source.

BSPL is different from continuous, non-pulsed UV light in a number of ways. The spectrum of BSPL contains UV light, but also includes a broader light spectrum, in particular between about 170 nm and about 2600 nm. The spectrum of BSPL is similar to that of sunlight at sea level, although it is 90,000 times more intense, and includes UV wavelengths between 200 and 300 nm which are normally filtered by the earth's atmosphere. BSPL is applied in short durations of relatively high power, as compared to the longer exposure times and lower power of non-pulsed UV light.

Furthermore, in preferred embodiments, at least 1% (preferably at least 5% or at least 10% and more preferably at least 50%) of the energy density or fluence level of the pulsed polychromatic (or monochromatic) light emitted from the flashlamp is concentrated at wavelengths within a range of 200 nm to 320 nm. The duration of the pulses of the pulsed light should be approximately from about 0.01 ms to about 100 ms, for example, about 10 ms to 300 ms.

Treatment System

As a result of such illumination, pathogens, such as microorganisms, fungus, bacteria, contained within the fluid may be effectively deactivated up to a level of 6 to 7 logs reduction or more (i.e., a microbial reduction level that is commonly accepted as sterilization). Advantageously, it has been found by the inventors herein that the use of short duration, pulsed light, such as pulsed polychromatic light and broad-spectrum pulsed light (i.e., BSPL), effectively reduces the treatment time or exposure time of the treatment of fluids significantly (e.g., about 2 to 20 seconds compared to several minutes or more), increases the deactivation rate of microorganisms on objects to a level commonly accepted as sterilization (about greater than 6 logs reduction of compared to 2-4 logs reduction), in comparison to known continuous wave UV fluid treatment systems.

Several apparatus designed to provide high-intensity, short duration pulsed incoherent polychromatic light in a broad-spectrum are described, for example, in U.S. Pat. Nos. 4,871,559 of Dunn, et al., entitled METHODS FOR PRESERVATION OF FOODSTUFFS, issued Oct. 3, 1989; U.S. Pat. No. 4,910,942 of Dunn, et al., entitled METHODS FOR ASEPTIC PACKAGING OF MEDICAL DEVICES, issued Mar. 27, 1990; U.S. Pat. No. 5,034,235 of Dunn, et al., entitled METHODS FOR PRESERVATION OF FOODSTUFFS, issued Jul. 23, 1991; U.S. Pat. No. 5,489,442 of Dunn, et al., entitled PROLONGATION OF SHELF LIFE IN PERISHABLE FOOD PRODUCTS, issued Feb. 6, 1996; U.S. Pat. No. 5,768,853 of Bushnell, et al., entitled DEACTIVATION OF MICROORGANISMS, issued Jun. 23, 1998; U.S. Pat. No. 5,786,598 of Clark, et al., entitled STERILIZATION OF PACKAGES AND THEIR CONTENTS USING HIGH-DENSITY, SHORT-DURATION PULSES OF INCOHERENT POLYCHROMATIC LIGHT IN A BROAD SPECTRUM, issued Jul. 28, 1998; U.S. Pat. No. 5,900,211 of Dunn, et al., entitled DEACTIVATION OF ORGANISMS USING HIGH-INTENSITY PULSED POLYCHROMATIC LIGHT, issued May 4, 1999; U.S. Provisional Application No. 60/291,850, of Fries et al., entitled SYSTEM FOR DECONTAMINATION OF FLUID PRODUCTS USING BROAD SPECTRUM LIGHT, filed May 17, 2001; U.S. application Ser. Nos. 09/976,597 and 09/976,776, both entitled SYSTEM FOR DECONTAMINATION OF FLUID PRODUCTS USING BROAD SPECTRUM LIGHT, and both filed Oct. 12, 2001, all of which are assigned to PurePulse Technologies of San Diego, California and all of which are incorporated herein by reference.

The following examples illustrate methods for carrying out the invention and should be understood to be illustrative of, but not limiting upon, the scope of the invention which is defined in the appended claims.

### EXAMPLES

### Example 1

### Inactivation of E. coli in Platelets

E. coli was added to concentrated platelets and the E. coli platelet mixture was diluted 1:6, 1:24 and 1:64 in PAS III. Dilutions at a 5 mm depth were illuminated with between 0-4 J/cm² total energy using fluences of 0.05, 0.25 and 0.5 J/F.

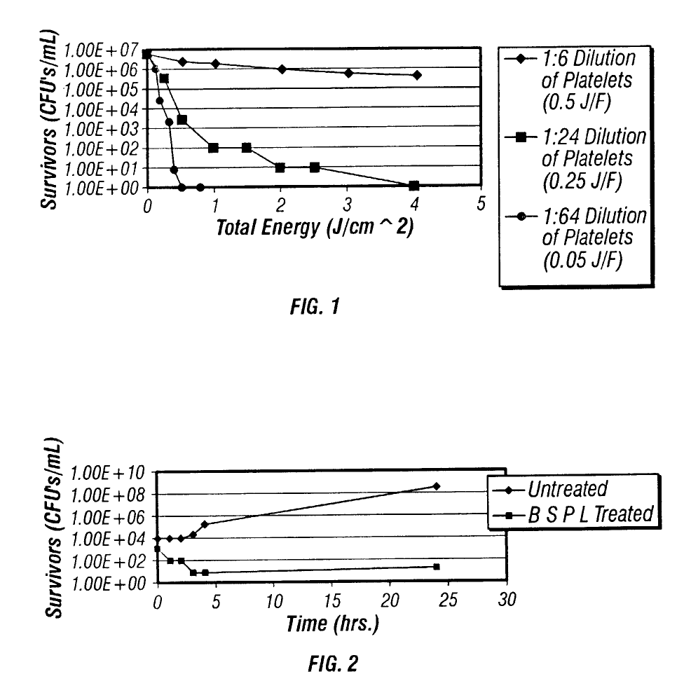

As illustrated in FIG. 1, BSPL was effective for reducing the concentration of E. coli in diluted platelet solutions exposed to various levels of BSPL.

### Example 2

### Periodic Treatment of Platelets

S. epidermis was added to concentrated platelets and the mixture was illuminated with 10 flashes of 0.5 J/F BSPL at 0, 1, 2, 3 and 4 hours in a 1.5 mm flat plate. Microbial counts were conducted at 0, 1, 2, 3, 4 and 24 hours.

As illustrated in FIG. 2, periodic BSPL treatment was effective for reducing and maintaining a reduction in the concentration of S. epidermis in concentrated platelet solutions exposed to BSPL.

### Example 3

### Periodic Treatment of Platelets

Various amount of S. epidermis were added to concentrated platelets and the mixture was illuminated with 10 flashes of 0.25 J/F BSPL every 2 hours.

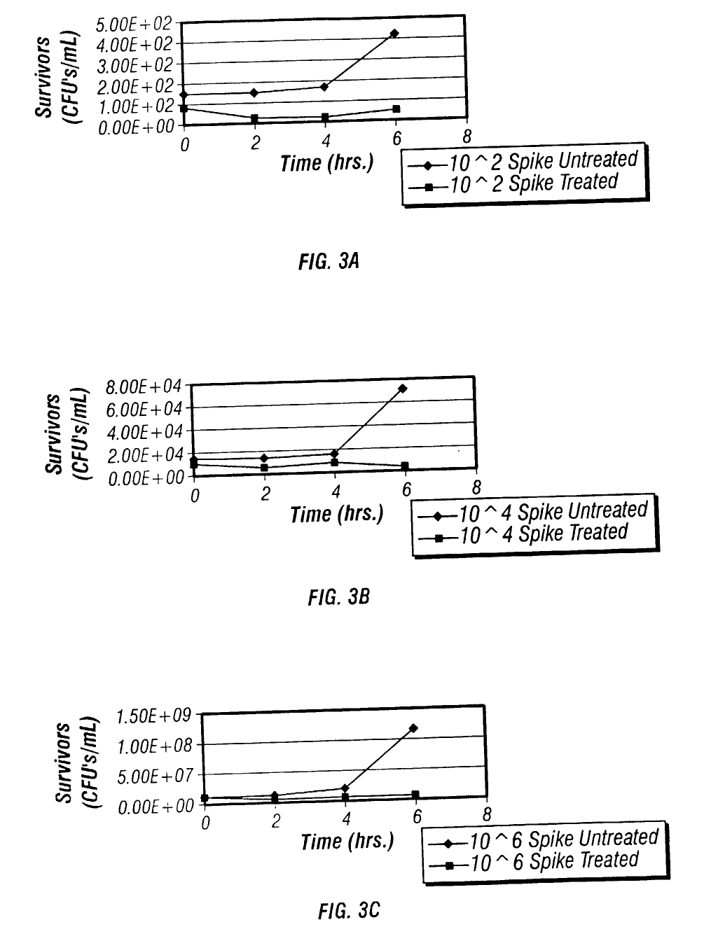

As illustrated in FIG. 3a-c, periodic BSPL treatment was effective for maintaining reduced levels of S. epidermis in platelet solutions exposed to BSPL treatment over time.

### Example 4

### Aggregation of Platelets

Platelets diluted 1:10 in PAS II solution were illuminated with 0-10 J/cm² total energy at 0.25 J/F. Samples were reconstituted 10× in Fetal Bovine Serum before aggregation analysis in response to collagen.

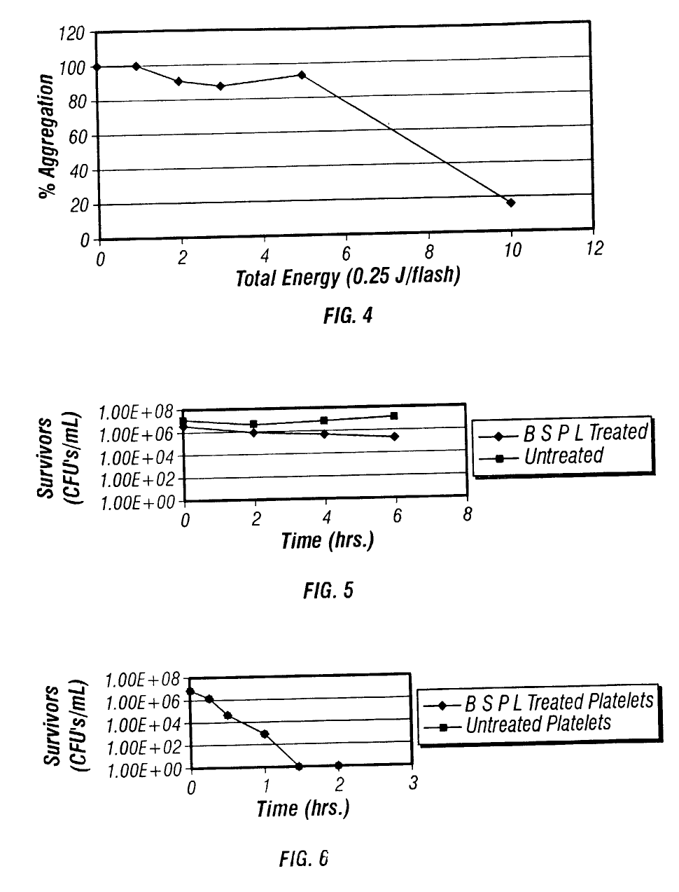

As illustrated in FIG. 4, BSPL treatment of a 1:10 dilution of platelets did not result in significant aggregation at energy level exposures that inactivate microbes.

### Example 5

### Inactivation of Microbes in Platelets

S. epidermidis was added to undiluted platelets and the mixture was treated with 5 J of energy (10 flashed at 0.5 J/F) every 2 hours. Numbers of surviving cells and % aggregation was determined over time.

As illustrated in FIG. 5a, S. epidermis levels did not increase in BSPL treated platelets over 6 hours.

### Example 6

### Continuous Treatment of Platelets

S. epidermidis was added to platelet solutions diluted 6-10× and the mixture was treated in a flow through system at a 1 mm sample depth at a flow rate of 100 ml/min. BSPL treatment was with 0.22 J/F with 2.0 J/cm² total enegy.

As illustrated in FIG. 6, the number of surviving microbes decreased with increasing energy. As further illustrated in the following table under treatment conditions yielding a >6 log reduction in number of surviving microbes, % aggregation only decreased by 21% % aggregation decreased with increasing total energy.

| Total | | Logs | |
|---|---|---|---|
| Energy | Fluence/Flash | Killed | % Aggregation |
| 0 | 0 | 0 | 100 |
| 2 | 0.22 | 6.47 | 79 |

### Example 7

### Effect of BSPL on Platelet Aggregation

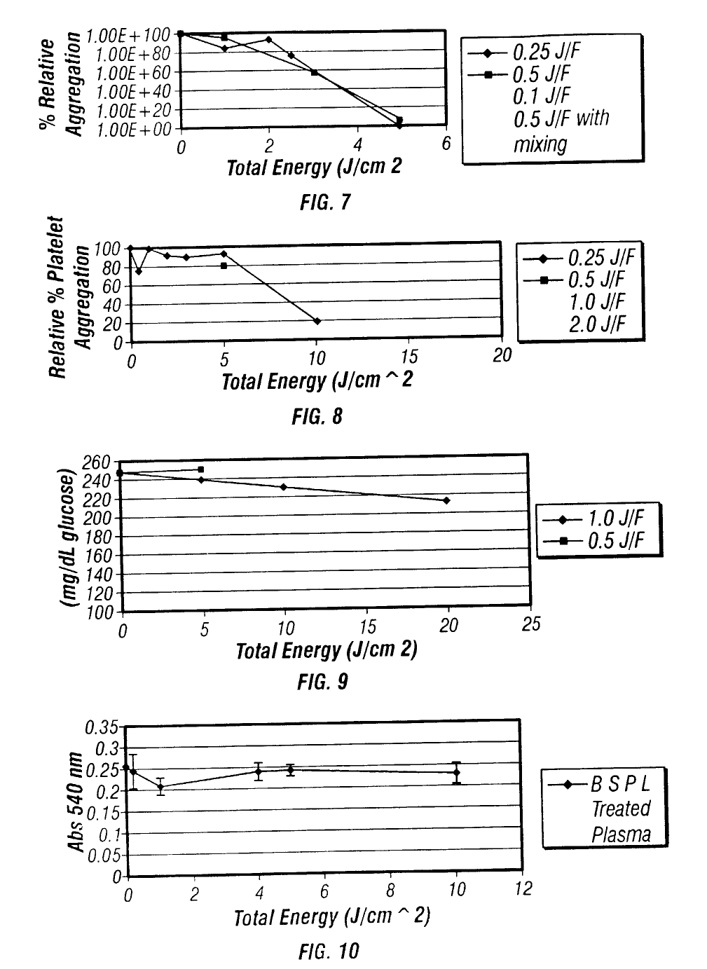

Platelets were diluted 1:10 in PAS III and exposed to 0-5 J/cm² total energy at 0.1, 0.25, 0.25 with mixing, and 0.5 J/F in a 5 mm sample depth. FIG. 7 shows the effects of BSPL on platelet function with and without mixing as well as a fluence/flash effect. Following reconstitution and after treatment, aggregation was determined. FIG. 8 shows the effects of reconstitution on platelet aggregation.

### Example 8

### Effect of Continuous BSPL Treatment on Platelet Aggregation

Platelet rich plasma was diluted 1:10 in PAS III, was run through the IFS-1 system at 100 mL/min, 0.22 J/F times 9 Flashes (2.0 J/cm² total energy), at a 1 mm sample depth. Results were as follows:

| Sample | Total Energy | Logs Killed | % Aggregation |
|---|---|---|---|
| Untreated | 0 | 0 | 100 |
| Treated | 2 | 6.47 | 79 |

### Example 9

### Effect on Platelet Physiology

Samples were diluted 1:10 in PAS III and exposed to 0-20 J/cm² total energy. Samples were examined for plasma glucose levels as a marker of cell integrity in accordance with Sigma Assay #115. Results are shown in FIG. 9. Platelets that lyse lose ATP and ATP breaks down sugar which in turns lowers plasma glucose levels.

### Example 10

### Effect on Platelet Physiology

Samples were diluted 1:10 in PAS III and exposed 0-10 J/cm² total energy. Samples were examined for plasma lactic acid levels as a marker of carbohydrate metabolism in accordance with Sigma Assay #735. Results are shown in FIG. 10.

### Example 11

### Effect of BSPL on Cell Function

Recovery of beta-galactosidase (b-gal) activity from E. coli cells exposed to BSPL demonstrates the ability to inactivate the E. coli cells but recover active proteins or enzymes. Since b-gal is located inside the cytoplasmic membrane of E. coli, the experiment demonstrates that after BSPL treatment the membrane does not become porous to all small molecules and retains it's barrier function. This is consistent with the observation that the cells remain phase dark when viewed in wet mounts by phase microscopy and supports the principle that whole cells (or virus particles) can be inactivated and used to prepare antigens for vaccines with the non-nucleic acid components.

E. coli cells (ATCC 1175) were grown on culture medium with and without the addition of lactose. No glucose was added to the medium. The cultures were diluted to approximately 6-7 logs of viable cell counts. Samples were exposed to increasing amounts of BSPL. Viability was measured by determining the number of colony forming units before and after exposure to BSPL. Recovery of b-gal was determined before and after exposure to BSPL by disrupting the membrane with toluene and sodium deoxycholate to allow ONPG to diffuse rapidly into the cytoplasm and come in contact with the b-gal. It was thought that ONPG would diffuse or be transported slowly through intact membranes unless a specific transport system is induced to move ONPG more rapidly into the cytoplasm. Incubation of the cells with ONPG was for 16 h before measuring the amount of enzyme activity.

As shown in Table 1, cells grown in the presence of lactose were completely killed by increasing exposure to BSPL. However, a corresponding proportional loss of b-gal was not observed. This means BSPL inactivated the cellular ability to divide by damaging nucleic acids but did not inactivate b-gal, a representative protein. This proves that BSPL can be used to selectively inactivate nucleic acids to the extent cell division does not occur, but useful proteins can be recovered intact and active. Similar results were obtained for cells grown in the absence of lactose (Table 2).

A comparison of lactose induced and non-induced cells is presented in Table 3. Both cell types were exposed to 0.25 j/m² BSPL completely eliminating survivors. Recovery of b-gal activity was measured in the cells by adding ONPG and incubating for 16 h. However, in these samples the cytoplasmic membrane was not disrupted by toluene and sodium deoxycholate. In the cells that were not induced with lactose, very little b-gal was recovered because the ONPG did not penetrate the cytoplasmic membranes and come in contact with the b-gal located in the cytoplasm. On the other hand, the induced cells allowed ONPG to penetrate the membrane and come in contact with the b-gal. This means the transport system of the E. coli membranes induced by lactose to more effectively bring lactose and similar compounds such as ONPG were not damaged by the BSPL. In the absence of induction by ONPG, the membrane remains a barrier to ONPG after exposure to BSPL. The ability to inactivate a cell and retain membrane barrier function and cellular antigens is valuable to inactivate microbial contaminants in platelet solutions. Platelets do not contain nucleic acids and their membrane must remain functionally intact, preserving all cellular antigens involved in the clotting cascade, for therapeutic purposes.

TABLE 1
(with lactose)

| | | | % B- |
|---|---|---|---|
| Energy | log 10 | log | galactosidase |
| (J/cm²) | Survivors | reduction | recovery |
| 0 | 7.01 | 0 | 100 |
| 0.05 | 6.09 | 0.92 | 97 |
| 0.1 | 5.12 | 1.89 | 91 |
| 0.15 | 3.66 | 3.35 | 88 |
| 0.2 | 1 | 6.01 | 86 |
| 0.25 | <1 | >6.01 | 97 |

TABLE 2
(without lactose)

| | | | % B- |
|---|---|---|---|
| Energy | log 10 | log | galactosidase |
| (J/cm²) | Survivors | reduction | recovery |
| 0 | 6.88 | 0 | 100 |
| 0.05 | 5.75 | 1.13 | 100 |
| 0.1 | 3.51 | 3.37 | 91 |
| 0.15 | <1 | >5.88 | 81 |
| 0.2 | <1 | >5.88 | 79 |
| 0.25 | <1 | >5.88 | 71 |

TABLE 3
(assay without disrupting membrane)

| | | | | % B |
|---|---|---|---|---|
| | | | | galactosidaseactivity |
| Energy | log 10 | log | Lactose | of control - toluene |
| (J/cm²) | Survivors | reduction | Present | & sodium deoxycholate |
| 0.25 | <1 | >5.88 | NO | 13% |
| 0.25 | <1 | >6.01 | YES | 51% |

Numerous modifications and variations in practice of the invention are expected to occur to those skilled in the art upon consideration of the foregoing detailed description of the invention. Consequently, such modifications and variations are intended to be included within the scope of the following claims.

What is claimed is:

1. A method of inactivating microbes in a platelet composition, the method comprising illuminating the biological fluid with pulses of a light having at least one wavelength within a range of 170 to 2600 nm and a fluence greater than about 0.001 J/cm², the illumination effective for inactivating microbes in the platelet composition and for decreasing platelet aggregation by not more than about 40%.

2. The method of inactivating microbes of claim 1 wherein the platelet composition is illuminated with pulses of light having wavelengths within a spectral range of at least between about 240 nm and about 280 nm and having a pulse duration of less than 100 ms.

3. The method of inactivating microbes of claim 1 wherein the platelet composition is selected from the group consisting of platelet rich plasma, leukocyte reduced platelets, non-leukocyte reduced platelets, synthetic platelet substitutes, artificial platelets, recombinant platelet products, and mixtures thereof.

4. The method of inactivating microbes of claim 1 wherein the biological fluid is illuminated with an amount of light effective for providing a fluence level of about 0.1 to about 0.6 J/cm².

5. A method of inactivating microbes in a platelet composition, the method comprising illuminating the platelet solution with pulses of a light having at least one wavelength within a range of 170 to 2600 nm and a fluence level of about 0.05 to about 15 J/cm², the illumination effective for inactivating microbes in the platelet composition by at least about 2 logs, and for decreasing platelet aggregation by not more than about 40%.

6. The method of inactivating microbes of claim 5 wherein the platelet composition is illuminated with pulses of light having wavelengths within a spectral range of at least between about 240 nm and about 280 nm and having a pulse duration of less than about 100 ms.

7. The method of inactivating microbes of claim 5 wherein the platelet composition is selected from the group consisting of platelet rich plasma, leukocyte reduced platelets, non-leukocyte reduced platelets, synthetic platelet substitutes, artificial platelets, recombinant platelet products, and mixtures thereof.

8. A method of inactivating microbes in a platelet composition, the method comprising:
flowing the platelet composition through a treatment chamber, the treatment chamber being light transmissive to at least 1% of a light treatment having at least one wavelength within a range of 170 to 2600 nm;
illuminating the platelet composition with the light as the platelet composition is flowed through the flexible treatment chamber;
inactivating microbes within the platelet composition, the method effective for inactivating microbes in the platelet composition by at least about 2 logs, and for decreasing platelet aggregation by not more than about 40%.

9. The method of inactivating microbes of claim 8 wherein the illuminating step comprises illuminating the platelet composition with pulses of light.

10. The method of inactivating microbes of claim 8 wherein the platelet composition is illuminated with pulses of light having wavelengths within a spectral range of at least between about 240 nm and about 280 nm and having a pulse duration of less than 100 ms.

11. The method of inactivating microbes of claim 8 wherein at least 1% of the fluence of the pulses of light is concentrated at wavelengths within a range of 200 to 300 nm.

12. The method of inactivating microbes of claim 8 wherein the platelet composition is flowed through the treatment chamber at a constant flow rate.

13. The method of inactivating microbes of claim 8 wherein the biological fluid is selected from the group consisting of platelet rich plasma, leukocyte reduced platelets, non-leukocyte reduced platelets, synthetic platelet substitutes, artificial platelets, recombinant platelet products, and mixtures thereof.

14. A method for increasing shelf-life of a platelet composition, the method comprising:
illuminating the platelet composition with pulses of a light having at least one wavelength within a range of 170 to 2600 nm and a fluence greater than about 0.001 J/cm², and repeating the illumination of the platelet composition every 6 hours, the illumination effective for inactivating microbes and for providing a zero net increase of microbial counts in the platelet composition, wherein platelet aggregation is not decreased by more than about 40%.

15. The method of inactivating microbes of claim 14 wherein the platelet composition is illuminated with pulses of light having wavelengths within a spectral range of at least between about 240 nm and about 280 nm and having a pulse duration of less than 100 ms.

16. The method of inactivating microbes of claim 14 wherein the platelet composition is illuminated with an amount of light effective for providing a fluence level of about 0.1 to about 0.6 J/cm².

17. A method for inactivating an endogenous nucleic acid strand, the method comprising illuminating an organisms containing the nucleic acid strand with at least one high-intensity, short duration pulse of incoherent polychromatic light in a broad spectrum.

18. The method according to claim 17, wherein the nucleic acid to be inactivated is endogenous and contained as part of a mammalian cell, a eukaryotic cell, plant cell, biological tissue, tumor cells, chloroplast, cellular organelle, virus, bacteria, fungi, phage, transposon, spores, vaccine, antigen, prion, vector, or mixtures thereof.

19. A method for inactivating microbes in a platelet composition, the method comprising:
illuminating the platelet composition with pulses of a light having at least one wavelength within a range of 170 to 2600 nm and a fluence greater than about 0.001 J/cm², and repeating the illumination of the platelet composition every 6 hours, the illumination effective for inactivating microbes and for providing a zero net increase of microbial counts in the platelet composition,
wherein platelet aggregation is not decreased by more than about 40%.

20. The method of inactivating microbes of claim 19 wherein the platelet composition is illuminated with pulses of light having wavelengths within a spectral range of at least between about 240 nm and about 280 nm and having a pulse duration of less than 100 ms.

21. The method of inactivating microbes of claim 19 wherein the platelet composition is illuminated with an amount of light effective for providing a fluence level of about 0.1 to about 0.6 J/cm².
